(19) United States
(12) Patent Application Publication
(10) Pub. No.: US 2003/0073705 A1
(43) Pub. Date: Apr. 17, 2003

(54) Method of treatment

(76) Inventors: Shahnaz Shahinfar, Newton Square, PA (US); Zhongxin Zhang, Blue Bell, PA (US); Barry M. Brenner, Weston, MA (US)

Correspondence Address: MERCK AND CO INC, P O BOX 2000, RAHWAY, NJ, 070650907

(21) Appl. No.: 10/143,415

(22) Filed: May 10, 2002

Related U.S. Application Data
(60) Provisional application No. 60/290,839, filed on May 14, 2001.

(51) Int. Cl. A61K 31/519; A61K 31/513; A61K 31/506; A61K 31/4745; A61K 31/4196

(52) U.S. Cl. 514/259.31; 514/269; 514/303; 514/381; 514/383

(57) ABSTRACT

This disclosure relates to a method of preventing end stage renal disease using an angiotensin II antagonist in patients with impaired renal function. Angiotensin II antagonists such as candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazolecarboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof are useful.

### BACKGROUND OF THE INVENTION

Angiotensin II (AII) is a potent vasoconstrictor. Its generation in the renin-angiotensin cascade results from the enzymatic action of renin on a blood plasma, 2-globulin, angiotensinogen, to produce angiotensin I (AI). AI is then converted by angiotensin converting enzyme (ACE) to the octapeptide hormone, AII. AII has been implicated as a causitive agent in hypertension. Therefore, ACE inhibitiors, which inhibit the production of AII, and AII receptor antagonists, which inhibit the function of AII, are useful in the treatment of hypertension. The efficacy of these compounds in the treatment of heart failure is also being studied.

Pals, et al., Circulation Research, 29, 673 (1971) describe the introduction of a sarcosine residue in position 1 and alanine in position 8 of the endogenous vasoconstrictor hormone AII to yield an (octa)peptide that blocks the effects of AII on the blood pressure of pithed rats. This analog, [Sar1, Ala8] AII, initially called “P-113” and subsequently “Saralasin,” was found to be one of the most potent competitive antagonists of the actions of AII, although, like most of the so-called peptide-AII-antagonists, it also possesses agonistic actions of its own. Saralasin has been demonstrated to lower arterial pressure in mammals and man when the (elevated) pressure is dependent on circulating AII (Pals et al., Circulation Research, 29, 673 (1971); Streeten and Anderson, Handbook of Hypertension, Vol. 5, Clinical Pharmacology of Antihypertensive Drugs, A. E. Doyle (Editor), Elsevier Science Publishers B. V., p. 246 (1984)). However, due to its agonistic character, saralasin generally elicits pressor effects when the pressure is not sustained by AII. Being a peptide, the pharmacological effects to saralasin are relatively short-lasting and are only manifest after parenteral administration, oral doses being ineffective. Although the therapeutic uses of peptide AII-blockers, like saralasin, are severely limited due to their oral ineffectiveness and short duration of action, their major utility is as a pharmaceutical standard.

Some known non-peptide antihypertensive agents act by inhibiting an enzyme, called angiotensin converting enzyme (ACE), which is responsible for conversion of angiotensin I to AII. Captopril and enalapril are commercially available ACE inhibitors (ACEI's). Based on experimental and clinical evidence, about 40% of hypertensive patients are non-responsive to treatment with ACEI's. But when a diuretic such as furosemide or hydrochlorothiazide is given together with a CEI, the blood pressure of the majority of hypertensive patients is effectively normalized.

Diuretic treatment converts the non-renin dependent state in regulating blood pressure to a renin-dependent state. Although All antagonist compounds act by a different mechanism, i.e., by blocking the AII receptor rather than by inhibiting the angiotensin converting enzyme, both mechanisms involve interference with the renin-angiotensin cascade. A combination of the ACEI enalapril maleate and the diruetic hydrochlorothiazide is commercially available under the trademark Vaseretic® from Merck & Co. Publications which relate to the use of diuretics with ACEI's to treat hypertension, in either a diuretic-first, stepwise approach or in physical combination, include Keeton, T. K. and Campbell, W. B., Pharmacol. Rev., 31:81 (1981) and Weinberger, M. H., Medical Clinics N. America, 71:979 (1987). Diuretics have also been administered in combination with saralasin to enhance the antihypertensive effect.

Losartan potassium (losartan) represents the first antihypertensive in the class of AII receptor antagonists which is disclosed in a U.S. Pat. No. 5,138,069 issued on Aug. 11, 1992, and which is assigned to E.I. du Pont de Nemours. Losartan has been demonstrated to be a potent orally active AII antagonist, selective for the AT1 receptor subtype useful in the treatment of hypertension. U.S. Pat. No. 5,210,079 issued to E.I. du Pont de Nemours and Company discloses the use of losartan as useful in the treatment of chronic renal failure.

Additionally, angiotensin II antagonists including losartan, have been disclosed as useful in 1) the treatment of hyperuricemia [U.S. Pat. No. 5,260,322 expires in October 2011.]; 2) the prevention of N-SAID induced renal failure U.S. Pat. No. 5,155,118 expires in October 2009; 3) to increase the survival rate of transplant patients, including renal and heart transplant patients [WO 97/02032]; 4) to improve insulin sensitivity [U.S. Pat. No. 5,962,500 expires March 2015]; and 5) to maintain glomerular filtration rate while inhibiting extracellular matrix accumulation [U.S. Pat. No. 5,512,580].

Interruption of the renin-angiotensin system with angiotensin I converting enzyme inhibitors significantly slows the progression of renal disease, both in Type 1 diabetic and nondiabetic patients with overt nephropathy. See 1. Lewis E J, Hunsicker L G, Bain R P, Rohde R D. The effect of angiotensin-converting-enzyme inhibition on diabetic nephropathy. N Engl J Med 1993;329:1456-62; and Maschio G, Alberti D, Janin G, et al. Effect of the angiotensin-converting-enzyme inhibitor benazepril on the progression of chronic renal insufficiency. N Engl J Med 1996;334:939-45; and The GISEN Group. Randomised placebo-controlled trial of effect of ramipril on decline in glomerular filtration rate and risk of terminal renal failure in proteinuric, non-diabetic nephropathy. Lancet 1997;349:1857-63. However, preventing or delaying end-stage renal disease per se, an especially important goal in Type 2 diabetes, the leading cause of chronic renal failure in many countries, remains unproven.

A study in Type 2 diabetic patients with nephropathy was undertaken to address this shortcoming. The primary aim of the study was to determine whether losartan, either alone or in combination with conventional antihypertensive therapy, reduces the number of patients with Type 2 diabetes experiencing a doubling of serum creatinine concentration, end stage renal disease, or death compared to placebo-treated patients (with or without conventional antihypertensive therapy). In addition, the effects of losartan versus placebo was assessed on the following secondary endpoints: composite of cardiovascular morbidity and mortality, proteinuria and progression of renal disease (slope of the reciprocal of serum creatinine concentration).

### SUMMARY OF THE INVENTION

This invention relates to a method of preventing end stage renal failure in patients with impaired renal function using an angiotensin II antagonist or a composition containing an angiotensin II antagonist. A second embodiment of the invention is a method of delaying the progression of renal disease in patients with impaired renal function. A third embodiment of the invention is a method of slowing the development of end stage renal disease in patients with nephropathy. Yet another aspect of the invention is a method of reducing hospitalization for heart failure in patients without clinical signs of heart failure, and who have impaired renal function.

### BRIEF DESCRIPTION OF THE DRAWINGS

FIG. 1. Trough Arterial Blood Pressure (Systolic, Diastolic and Mean) (Key: P denotes placebo; L denotes losartan; +CT denotes plus conventional therapy. Mean follow-up time was 3.4 years (42 months).)

FIG. 2. Kaplan Meier Curves of the Percentage of Patients with the Composite Primary Endpoint of Doubling of Serum Creatinine Concentration, End Stage Renal Disease or Death in the Losartan and Placebo Arms. (Key: P denotes placebo; L denotes losartan; +CT denotes plus conventional therapy. Mean follow-up time was 3.4 years (42 months).)

In the terminal months of the study, the losartan and placebo curves tended to converge. Such convergence is specific to one component of the primary endpoint, namely, doubling of serum creatinine concentration (FIG. 3A). This pattern, also seen previously in Type 1 diabetic patients [Lewis], may be due to the higher risk profile (i.e., higher baseline serum creatinine and urine protein concentrations) of those patients in the losartan group who remained event-free until month 40, as compared to the placebo group.

FIG. 3. Kaplan-Meier Curves of the Percentage of Patients with the Individual Components of the Primary Composite Endpoint in the Losartan and Placebo Arms. Panels A, B, and C depict percentage of patients with doubling of serum creatinine concentration, end stage renal disease, and the combined endpoint of end stage renal disease or death, respectively. P denotes placebo; L denotes losartan; +CT denotes plus conventional therapy. Mean follow-up time was 3.4 years (42 months).

FIG. 4. Kaplan-Meier Curves of the Percentage of Patients Who Were Hospitalized for Heart Failure in the Losartan and Placebo Arms. Curves represent time to first hospitalization. Subsequent hospitalizations for heart failure were not assessed. P denotes placebo; L denotes losartan; +CT denotes plus conventional therapy. Mean follow-up time was 3.4 years (42 months).

FIG. 5. Changes from Baseline in Proteinuria. Proteinuria was measured as the urine albumin:creatinine ratio in a first morning specimen. (Key: P denotes placebo; L denotes losartan; +CT denotes plus conventional therapy. Mean follow-up time was 3.4 years (42 months).)

FIG. 6. Effect of Losartan on the Primary Composite, End Stage Renal Disease, and Combined End Stage Renal Disease or Death Endpoints in Various Baseline Subgroups. For each subgroup, the hazard ratio with losartan is plotted. Horizontal lines represent 95% confidence intervals. (Key: sCr denotes serum creatinine; ESRD denotes end stage renal disease; SBP denotes systolic blood pressure; UA:CR denotes urine albumin:creatinine ratio; HbA1c denotes glycosylated hemoglobin; CCB denotes calcium channel blockers (dihydropyridine calcium channel antagonists); ACEI/AIIA denotes angiotensin I converting enzyme inhibitors/angiotensin II receptor antagonists; P denotes placebo; L denotes losartan.)

### DETAILED DESCRIPTION OF THE INVENTION

A method of preventing end stage renal failure in patients with impaired renal function comprising administration of a therapeutically effective amount of an angiotensin II antagonist or a composition containing an angiotensin II antagonist.

The method as recited above, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl) [1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

The method as recited above, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

The method as recited above, wherein the composition containing an angiotensin II antagonist includes hydrochlorothiazide.

The method as recited above, wherein the angiotensin II antagonist is losartan potassium or a composition containing losartan potassium.

The method as recited above, wherein the patient is a diabetic patient.

The method as recited above, wherein the patient is a Type II diabetic patient.

The method as recited above, wherein the patient is a renal transplant patient.

A method of delaying the progression of renal disease in patients with impaired renal function comprising administration of a therapeutically effective amount of an angiotensin II antagonist or a composition containing an angiotensin II antagonist.

The method as recited above, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl) [1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

The method as recited above, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

The method as recited above, wherein the composition containing an angiotensin II antagonist includes hydrochlorothiazide.

The method as recited above, wherein the angiotensin II antagonist is losartan potassium or a composition containing losartan potassium.

The method as recited above, wherein the patient is a diabetic patient.

The method as recited above, wherein the patient is a Type II diabetic patient.

The method as recited above, wherein the patient is a renal transplant patient.

A method of slowing the development of end stage renal disease in patients with nephropathy comprising administration of a therapeutically effective amount of an angiotensin II antagonist.

The method as recited above, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl) [1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl)[1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5 -yl)biphenyl-4-yl]methyl]-1H-imidazole-5-carboxylic acid 1-ethoxycarbonyloxyethyl ester, or pharmaceutically acceptable salts thereof.

The method as recited above, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

The method as recited above, wherein the composition containing an angiotensin II antagonist includes hydrochlorothiazide.

The method as recited above, wherein the angiotensin II antagonist is losartan potassium or a composition containing losartan potassium.

The method as recited above, wherein the patient is a diabetic patient.

The method as recited above, wherein the patient is a Type II diabetic patient.

The method as recited above, wherein the patient is a renal transplant patient.

A method of reducing hospitalization for heart failure in patients without clinical signs of heart failure, and who have impaired renal function comprising administration of a therapeutically effective amount of an angiotensin II antagonist.

The method as recited above, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo [1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

The method as recited above, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

The method as recited above, wherein the composition containing an angiotensin II antagonist includes hydrochlorothiazide.

The method as recited above, wherein the angiotensin II antagonist is losartan potassium or a composition containing losartan potassium.

The method as recited above, wherein the patient is a diabetic patient.

The method as recited above, wherein the patient is a Type II diabetic patient.

The method as recited above, wherein the patient is a renal transplant patient.

End stage renal failure ultimately results in the death of the patient's kidneys. The patient's life is in many countries can be prolonged by artificial means through renal transplant and dialysis. However, in countries where these options are not available, end stage renal disease results in the death of the patient due to end stage renal failure. Impaired renal function is defined as a proteiniuria (patients having a urine albumin of 300 mg/g creatinine, or a 24-hour urine protein level of >=500 mg (0.5 g/day), and serum creatinine values between 1.3 and 3.0 mg/dL (lower limit of 1.5 mg/dL for males >60 kg).

The compounds of the present invention are AT1 selective, angiotensin II antagonists that can be prepared using procedures described in the art. Additionally, a number of the commercially available angiotensin II antagonists are noted below:

| Generic | | |
|---|---|---|
| Name | Patent No. | Chemical name |
| Candesartan | U.S. Pat. No. | (+/−)-1-(cyclohexylcarbonyloxy)ethyl-2- |
| | 5,703,110 | ethoxy-1-[[2′-(1H-tetrazol-5-yl)biphenyl-4- |
| | U.S. Pat. No. | yl]-1H-benzimidazole-7-carboxylate |
| | 5,196,444 | |
| Eprosartan | U.S. Pat. No. | 3-[1-(4-carboxyphenylmethyl)-2-n-butyl- |
| | 5,185,351 | imidazol-5-yl]-(2-thienylmethyl)-2- |
| | U.S. Pat. No. | propenoic acid |
| | 5,650,650 | |
| Irbesartan | U.S. Pat. No. | 2-n-butyl-3-[[2′-(1h-tetrazol-5-yl)biphenyl- |
| | 5,270,317 | 4-yl]methyl]1,3-diazazspiro[4,4]non-1-en- |
| | U.S. Pat. No. | 4-one |
| | 5,352,788 | |
| Losartan | U.S. Pat. No. | 2-N-butyl-4-chloro-5-hydroxymethyl-1- |
| | 5,138,069 | [(2′-(1H-tetrazol-5-yl)biphenyl-4-yl)- |
| | U.S. Pat. No. | methyl]imidazole, potassium salt |
| | 5,153,197 | |
| | U.S. Pat. No. | |
| | 5,128,355 | |
| Tasosartan | U.S. Pat. No. | 5,8-dihydro-2,4-dimethyl-8-[(2′-(1H- |
| | 5,149,699 | tetrazol-5-yl)[1,1′-biphenyl]4-yl)methyl]- |
| | | pyrido[2,3-d]pyrimidin-7(6H)-one |
| Telmisartan | U.S. Pat. No. | 4′-[(1,4-dimethyl-2′-propyl-(2,6′-bi-1H- |
| | 5,591,762 | benzimidazol)-1′-yl)]-[1,1′-biphenyl]-2- |
| | | carboxylic acid |
| Valsartan | U.S. Pat. No. | (S)-N-valeryl-N-[[2′-(1H-tetrazol-5- |
| | 5,399,578 | yl)biphenyl-4-yl]methyl]valine |
| EXP-3174* | U.S. Pat. No. | 2-N-butyl-4-chloro-1-[(2′-(1H-tetrazol-5- |
| | 5,138,069 | yl)biphenyl-4-yl)-methyl]imidazole-5- |
| | U.S. Pat. No. | carboxylic acid |
| | 5,153,197 | |
| | U.S. Pat. No. | |
| | 5,128,355 | |

*EXP3174 is an active metabolite of losartan.

The compounds useful in herein can be administered orally, intravenously, parentally, subcutaneously, etc. The preferred mode of administration for these compounds is oral administration. The dosage will depend of the potency of the angiotensin II antagonist being used; the dosage range is about 1.0 mg to about 1,000 mg, and preferably about 4 mg to about 600mg. The angiotensin II antagonists currently available on the market have been approved at the following doses:

| Generic Name | Approved Doses |
|---|---|
| Candesartan | 4, 8, 16, 32 mg |
| Eprosartan | 300, 400, 600 mg |
| Irbesartan | 75, 150, 300 mg |
| Losartan | 25, 50 mg |
| Telmisartan | 20, 40, 80 mg |
| Valsartan | 80, 160 mg |

Also within the scope of this invention is a composition containing an angiotensin II antagonist with hydrochlorothiazide (abbreviated HCTz) at a dosage range of about 6.25 mg to about 25 mg. Examples of such compositions that are currently available on the market are:

| AIIA Composition | Approved Doses |
|---|---|
| Candesartan/HCTz | 16 mg/12.5 mg |
| | 32 mg/12.5 mg |
| Irbesartan/HCTz | 150 mg/12.5 mg |
| | 300 mg/12.5 mg |
| Losartan/HCTz | 50 mg/12.5 mg |
| | 100 mg/25 mg |
| Telmisartan/HCTz | 40 mg/12.5 mg |
| | 80 mg/12.5 mg |
| Valsartan/HCTz | 80 mg/12.5 mg |
| | 160 mg/12.5 mg |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein may be applicable as a consequence of variations in the responsiveness of the patient being treated for any of the indications with the compounds of the invention indicated above. Likewise, the specific pharmacological responses observed may vary according to and depending upon the particular angiotensin II antagonist compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims, which follow, and that such claims be interpreted as broadly as is reasonable.

### EXAMPLE

### Study Design

This triple-blind, randomized, placebo-controlled study evaluated the renal protective effects of losartan in 1513 patients with Type 2 diabetes and nephropathy. The results reported here represent a mean follow-up time of 3.4 years.

### Patients

The study involved male and female patients ranging in age from 31 to 70 years diagnosed with Type 2 diabetes and nephropathy; the latter defined as two urine albumin:creatinine ratios from a first morning specimen >300 mg/g (34 mg/mmol) (or a 24-hr urine protein >=500 mg (0.5 g/day)) and two serum creatinine values between 1.3 and 3.0 mg/dL (115 and 265 μmol/L) (lower limit of 1.5 mg/dL (133 μmol/L)for males >60 kg). See Brenner B M, Cooper M E, de Zeeuw D, et al. The losartan renal protection study—rationale, study design and baseline characteristics of RENAAL (reduction of endpoints in NIDDM with the angiotensin II antagonist losartan). Journal of the Renin Angiotensin Aldosterone System 2000;1(4):328-35. Patients were excluded from the study if diagnosed with Type 1 diabetes or nondiabetic renal disease, including renal artery stenosis. Patients with a history of heart failure, myocardial infarction or coronary artery bypass grafting within 1 month, cerebral vascular accident or percutaneous transluminal coronary angioplasty within 6 months or transient ischemic attacks within 1 year prior to enrollment were excluded.

### Treatment

During the 6-week screening phase, hypertensive patients continued to take their standard antihypertensive therapy. However, in those patients taking angiotensin I converting enzyme inhibitors or angiotensin II receptor antagonists, these agents were discontinued at the beginning of the screening phase and replaced by alternative open-label therapy at the investigator's discretion (diuretic, calcium-channel antagonist, α- or β-blocker, and/or centrally acting agent). Patients were stratified by level of baseline proteinuria (urine albumin: creatinine ratio above or below 2000 mg/g (226 mg/mmol)) and were randomized to either losartan 50 mg or matching placebo once daily on a background of conventional antihypertensive therapy. After 4 weeks of active treatment, the study drug was increased to losartan (100 mg once daily) or placebo if the trough sitting blood pressure measurement was above the goal of <140/<90 mmHg. After an additional 8 weeks, further antihypertensive therapy with agents described above (exclusive of angiotensin I converting enzyme inhibitors or angiotensin II receptor antagonists), were added or increased in dosage to achieve the goal trough sitting blood pressure.

Throughout the study, patients received standard medical care for treatment of diabetes, including routine measurements of HbAlc and fasting serum glucose concentrations. Study visits were scheduled every 3 months or more frequently if necessary for monitoring trough sitting blood pressure control, changes in laboratory measurements, adverse experiences and endpoints. Patients who discontinued early from blinded study therapy were followed every 3 months until study end for primary and secondary endpoints, blood pressure control and laboratory measurements. Those who could not return for clinic visits were contacted by telephone for endpoints of dialysis, renal transplantation or death.

### Outcome Measures

The primary efficacy parameter was a composite endpoint of time to the first event of doubling of serum creatinine concentration, end stage renal disease, or death. End stage renal disease was defined as the need for chronic dialysis or renal transplantation. The prespecified secondary endpoint, cardiovascular morbidity and mortality, was a composite of myocardial infarction, stroke, hospitalization for heart failure or unstable angina, coronary or peripheral revascularization, and death from cardiovascular-related causes. Analyses of the components of both the primary and secondary composite endpoints also were prespecified. Other secondary endpoints included progression of renal disease as assessed by the slope of the reciprocal of serum creatinine concentration and changes in proteinuria. (See Mitch W E, Walser M, Buffington G A, Lemann J Jr. A simple method of estimating progression of chronic renal failure. Lancet 1976;2:1326-8.) An independent, blinded endpoint committee adjudicated all potential clinical endpoints occurring from the time of randomization until study termination.

### Statistical Analysis

Analyses of the primary and secondary clinical endpoints were based on the intention-to-treat principle; all randomized patients were included from randomization through the study termination date (with the exception of 3 patients lost to follow-up). A supportive, per-protocol analysis excluded patients who violated the inclusion/exclusion criteria, and censored patients 14 days after permanent discontinuation of study medication. A Cox regression model, including baseline level of proteinuria as a stratification factor, was used to determine the hazard risk ratio (losartan relative to placebo) and 95% confidence interval. See Cox D R. Regression models and life tables (with discussion). Journal of the Royal Statistical Society 1972;34 Series B:187-220. The risk reduction was calculated as 100%×(1-hazard ratio). In analyses of nonfatal endpoints, patients who died were considered censored. Event curves are based on the Kaplan-Meier procedure. See Kaplan E L and Meier P. Non-parametric estimation from incomplete observations. Journal of the American Statistical Association 1958;53:457-81. The sample sizes below the Kaplan-Meier curves represent the sizes of the risk sets at various timepoints, i.e., the number of patients with sufficient follow-up who have not yet had an event. The impact of between-group differences in blood pressure control was examined by adding on-treatment mean arterial pressure as a time-varying covariate in the Cox model, and comparing the estimated effect of losartan from this model with that from the primary analysis.

The analyses of renal progression and changes in proteinuria were based on an on-treatment approach. For renal function, the slopes of the reciprocal of serum creatinine concentration were compared between the two treatment groups using a linear random effects model. Changes in proteinuria were compared between the two treatment groups, using a mixed-effects model with terms including treatment at each time point, and baseline proteinuria levels. See The mixed procedure. In: SAS Institute Inc., SAS/STAT® software; changes and enhancements through release 6.12. Carey, N. C.: SAS Institute Inc.; 1977. P573-701.

Due to one interim analysis using a stopping boundary based on an O'Brien-Fleming-type alpha spending function, a critical p-value of 0.048 was required for the primary hypothesis. See O'Brien P C and Fleming T R. A multiple testing procedure for clinical trials. Biometrics 1979; 35:549-56. For other outcomes, a p-value of less than 0.05 was considered to indicate statistical significance. All statistical tests were two-sided.

### RESULTS

1513 patients were randomized to receive either losartan or placebo once daily on a background of conventional antihypertensive therapy, but excluding angiotensin I converting enzyme inhibitors and angiotensin II receptor antagonists. The dose of losartan was 50 to 100 mg once daily with 71% of the patients receiving 100 mg. Baseline characteristics were similar in the two treatment groups (Table 1). Patients were followed for an average of 3.4 years. Table 2 outlines the main reasons for discontinuation from study treatment. More patients discontinued study treatment in the placebo group compared to the losartan group. The status of all patients (except for 3 patients in the losartan group who could not be contacted) in terms of dialysis, transplantation or death was determined.

TABLE 1
Baseline Characteristics

| | Losartan | Placebo (+CT) |
|---|---|---|
| | (n = 751) | (n = 762) |
| Age (year) | 60 + 7 | 60 + 7 |
| Male | 462 (61.5) | 494 (64.8) |
| Female | 289 (38.5) | 268 (35.2) |
| Race | | |
| Asian | 117 (15.6) | 135 (17.7) |
| Black | 125 (16.6) | 105 (13.8) |
| Caucasian | 358 (47.7) | 378 (49.6) |
| Hispanic | 140 (18.6) | 136 (17.8) |
| Other | 11 (1.5) | 8 (1.0) |
| Medical History | | |
| Hypertension, years | 9.4 + 9.0 | 9.2 + 9.2 |
| Diabetes, years | 15.2 + 8.0 | 15.0 + 8.1 |
| Body Mass Index* | 30 + 6 | 29 + 6 |
| Systolic Blood Pressure (mmHg) | 152 + 19 | 153 + 20 |
| Diastolic Blood Pressure (mmHg) | 82 + 10 | 82 + 11 |
| Angina Pectoris | 65 (8.7) | 75 (9.8) |
| Myocardial Infarction | 75 (10.0) | 94 (12.3) |
| Coronary Revascularization | 1 (0.1) | 1 (0.1) |
| Stroke | 0 | 1 (0.1) |
| Lipid Disorders | 234 (31.2) | 271 (35.6) |
| Anemia | 159 (21.2) | 166 (21.8) |
| Amputation | 65 (8.7) | 69 (9.1) |
| Neuropathy | 375 (50.0) | 379 (49.7) |
| Retinopathy | 494 (65.8) | 470 (61.7) |
| Laser therapy/photocoagulation | 48 (6.4) | 55 (7.2) |
| Smoking (current) | 147 (19.6) | 130 (17.0) |
| Laboratory | | |
| Urine albumin:creatinine (median, mg/g) | 1237 | 1261 |
| Urine albumin:creatinine (mg/g)† | 1875 + 1831 | 1743 + 1544 |
| Serum Creatinine (mg/dL)‡ | 1.9 + 0.4 | 1.9 + 0.5 |
| Serum Cholesterol (mg/dL)§ | | |
| Total | 227 + 56 | 229 + 55 |
| LDL | 142 + 47 | 142 + 45 |
| HDL | 45 + 16 | 45 + 15 |
| Serum Triglycerides (mg/dL)∥ | 212 + 180 | 225 + 200 |
| Serum Potassium (mEq/L)¶ | 4.6 + 0.4 | 4.6 + 0.5 |
| Serum Uric Acid (mg/dL)** | 6.7 + 1.7 | 6.7 + 1.6 |
| Hemolgobin (g/dL)†† | 12.5 ± 1.8 | 12.5 ± 1.8 |
| Glycosylated hemoglobin (%) | 8.5 + 1.6 | 8.4 + 1.6 |
| Prior Medications | | |
| ACEi or Angiotensin II antagonist | 405 (53.9) | 381 (50.0) |
| Diuretic | 442 (58.9) | 435 (57.0) |
| Dihydropyridine calcium antagonist | 406 (54.0) | 411 (54.0) |
| Non-dihydropyridine calcium antagonist | 162 (21.6) | 160 (21.0) |
| β-blocker | 139 (18.5) | 145 (19.0) |
| α-blocker | 180 (24.0) | 184 (24.2) |
| Insulin | 470 (62.5) | 456 (59.8) |
| Oral antidiabetic agents | 361 (48.0) | 381 (50.0) |
| Aspirin | 255 (34.0) | 244 (32.0) |
| Lipid lowering agents | 274 (36.4) | 275 (36.0) |

Data presented are:

n (%) number of patients (percent)

plus/minus values are mean±standard deviation

+CT denotes plus conventional therapy

ACEi denotes angiotensin I converting enzyme inhibitor

*Body mass index was calculated as weight (kg) divided by square of height (m)

†To convert to mg/mmol, multiply by 0.113

‡To convert to umol/L, multiply by 88.4

§To convert to mmol/L, multiply by 0.02586

∥To convert to mmol/L, multiply by 0.01129

¶To convert to mmol/L, multiply by 1.

** To convert to umol/L, multiply by 59.48.

††To convert to mmol/L, multiply by 0.6206.

p=NS (not significant, losartan versus placebo) for all parameters.

TABLE 2
Reasons for Discontinuation from Study Therapy

| | Losartan (+CT) | Placebo (+CT) |
|---|---|---|
| | n = 751 | n = 762 |
| | n (%) | n (%) |
| Discontinued for Any Reason | 341 (45.4) | 396 (52.0) |
| Deaths | 65 (8.7) | 67 (8.8) |
| Clinical Adverse Experience* | 128 (17.0) | 165 (21.6) |
| Laboratory Adverse Experience* | 21 (2.8) | 16 (2.1) |
| Withdrew Consent | 54 (7.2) | 63 (8.3) |
| Other | 68 (9.1) | 85 (11.2) |
| Lost-to-follow-up | 3 (0.4) | 0 |
| Most Common Adverse Experiences | | |
| (Clinical) Causing Discontinuation† | | |
| Heart Failure | 23 (3.0) | 45 (5.9) |
| End Stage Renal Disease | 16 (2.1) | 22 (2.8) |
| Myocardial Infarction | 7 (0.9) | 13 (1.7) |
| Stroke | 9 (1.2) | 8 (1.0) |
| Worsening renal insufficiency | 8 (1.1) | 8 (1.0) |
| Most Common Adverse Experiences | | |
| (Laboratory) | | |
| Serum Creatinine Increased | 12 (1.6) | 10 (1.3) |
| Hyperkalemia | 9 (1.2) | 3 (0.4) |

+CT denotes plus conventional therapy.
*See lower part of table for breakdown of most common adverse experiences that led to discontinuation of study therapy.
†At the investigator's discretion, study drug was discontinued in some patients when an endpoint was reached.

### Blood Pressure

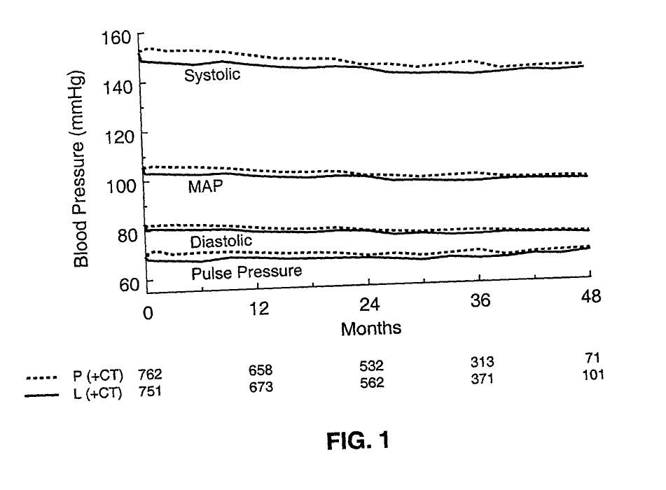

During baseline, 93.5% of patients were on antihypertensive therapy (92% in the losartan group versus 95% in the placebo group). Trough blood pressure fell progressively during the study (FIG. 1). Trough systolic/diastolic blood pressure at baseline averaged 153/82 mmHg in the placebo group and 152/82 in the losartan group (mean arterial pressure was 106.0 versus 105.5, p=0.38; pulse pressure was 70.8 versus 69.4, p=0.13). At 1 year, values averaged 150/80 and 146/78 (103.1 versus 100.9, p<0.001; 69.8 versus 67.8, p=0.047); at year 2, 144/77 and 143/77 (99.7 versus 99.1, p=0.38; 67.1 versus 66.2, p=0.37); and at study end, 142/74 and 140/74 (96.8 versus 95.9, p=0.59; 67.4 versus 66.7, p=0.77). Table 3 lists the various classes of conventional antihypertensive drugs utilized during the study.

TABLE 3
Concurrent Antihypertensive Medications*

| | Losartan (+CT) | Placebo (+CT) |
|---|---|---|
| | n = 751 | n = 762 |
| Therapeutic Class | n (%) | n (%) |
| Diuretic | 627 (83.5) | 640 (84.0) |
| Calcium Channel Antagonist | 657 (87.4) | 683 (90.0) |
| Dihydropyridine | 467 (61.3) | 491 (64.4) |
| Non-dihydropyridine | 199 (26.5) | 192 (25.2) |
| β-blocker | 303 (40.4) | 349 (45.8) |
| α-blocker | 255 (34.0) | 280 (36.8) |
| Centrally Acting Agent | 135 (18.0) | 167 (21.9) |

*Table comprises conventional antihypertensive therapy.
+CT denotes plus conventional therapy.

### Primary Outcomes

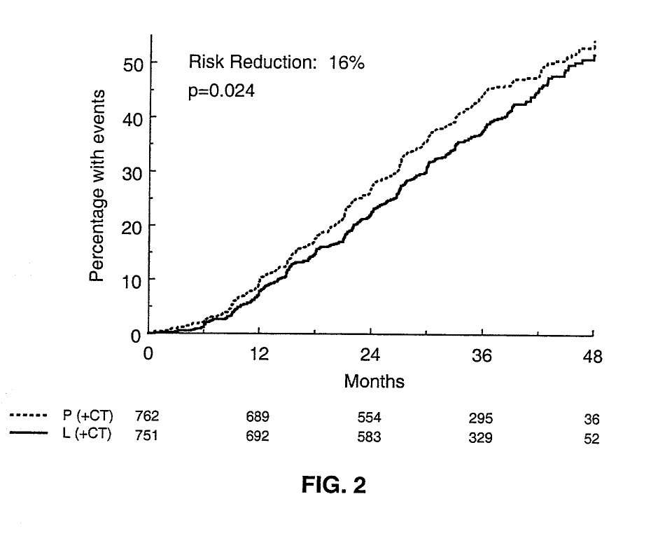

By the intention-to-treat analysis, the primary composite endpoint of doubling of serum creatinine concentration, end stage renal disease or death was reached in 327 (43.5%; 15.9/100 patient years of follow-up) patients given losartan versus 359 (47.1%; 18.1 per 100 patient years of follow-up) given placebo (FIG. 2). Losartan treatment resulted in a reduction of risk of 16% (p=0.024) in the primary composite endpoint. The decrease in risk remained essentially unchanged after correcting for blood pressure (15%, p=0.034). Furthermore, for those patients who remained on treatment throughout the protocol (per protocol analysis), losartan conferred a 22% (p=0.008) risk reduction in the primary composite endpoint.

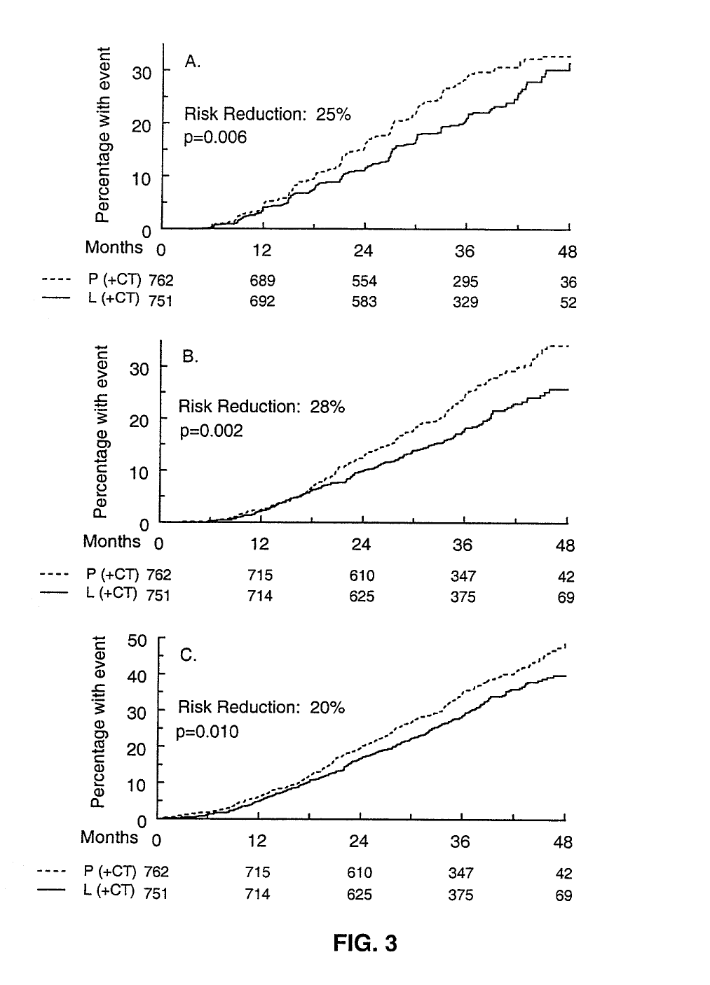

Analyses of the individual components of the primary endpoint are shown in Table 4. The risk of doubling of serum creatinine concentration was reduced by 25% (p=0.006) with losartan (FIG. 3A). Losartan also reduced the risk of end stage renal disease by 28% (p=0.002) (FIG. 3B). Approximately 20% of patients died with no difference between the two groups (p=0.882). However, it should be recognized that end stage renal disease and death are two potentially competing endpoints in that all deaths are tallied events whether or not the end stage renal disease endpoint is reached. As shown in FIG. 3C, the risk of this combined endpoint was reduced by 20% (p=0.01).

TABLE 4
Primary Endpoint and Components

| | Losartan | Placebo | | Risk | |
|---|---|---|---|---|---|
| | (+CT) | (+CT) | | Re- | |
| | (n = 751) | (n = 762) | | duction | |
| | n (%) | n (%) | p-Value | (%) | 95% CI |
| Doubling of sCr, | 327 (43.5) | 359 (47.1) | 0.024 | 16 | (2, 28) |
| ESRD or Death* | | | | | |
| Doubling of sCr | 162 (21.6) | 198 (26.0) | 0.006 | 25 | (8, 39) |
| ESRD | 147 (19.6) | 194 (25.5) | 0.002 | 28 | (11, 42) |
| Death | 158 (21.0) | 155 (20.3) | 0.882 | −2 | (−27, 19) |
| ESRD or Death | 255 (34.0) | 300 (39.4) | 0.010 | 20 | (5, 32) |
| Doubling of sCr | 226 (30.1) | 263 (34.5) | 0.010 | 21 | (5, 34) |
| or ESRD | | | | | |

*Composite primary endpoint.
ESRD denotes end stage renal disease
CI denotes confidence interval.
sCr denotes serum creatinine
+CT denotes plus conventional therapy

Secondary Outcomes

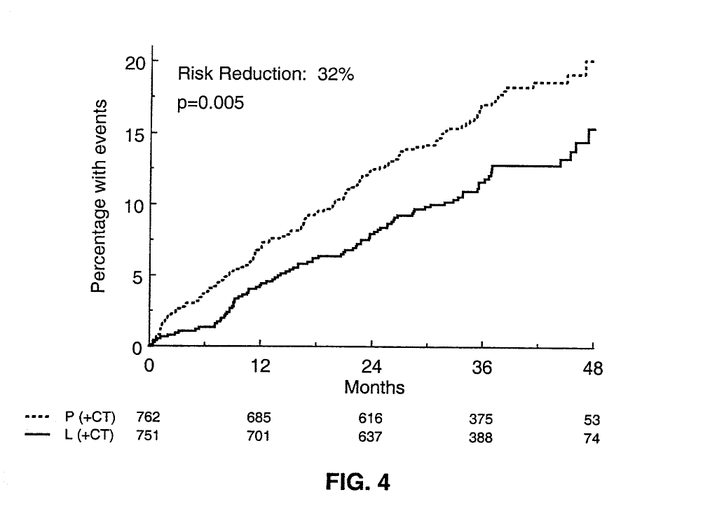

There was no significant difference between losartan plus conventional therapy and placebo plus conventional therapy for the composite endpoint of cardiovascular morbidity and mortality. Approximately one third of patients had a fatal or non-fatal cardiovascular event (245 with losartan and 266 with placebo, risk reduction=9%, p=0.26). No significant differences emerged with most of the cardiovascular components, except hospitalization for heart failure (89 patients with losartan versus 127 with placebo), of which the risk was reduced by 32% (p=0.005) (FIG. 4). There were 88 patients (44 in each group) randomized with preexisting heart failure. When excluding these patients from the analysis of this component, there remained a significant difference in hospitalization for heart failure between the two treatment groups. There was a difference in the number of myocardial infarctions between losartan and placebo, however, this difference did not reach statistical significance (50 patients with losartan versus 68 patients with placebo, risk reduction=28%, p=0.08).

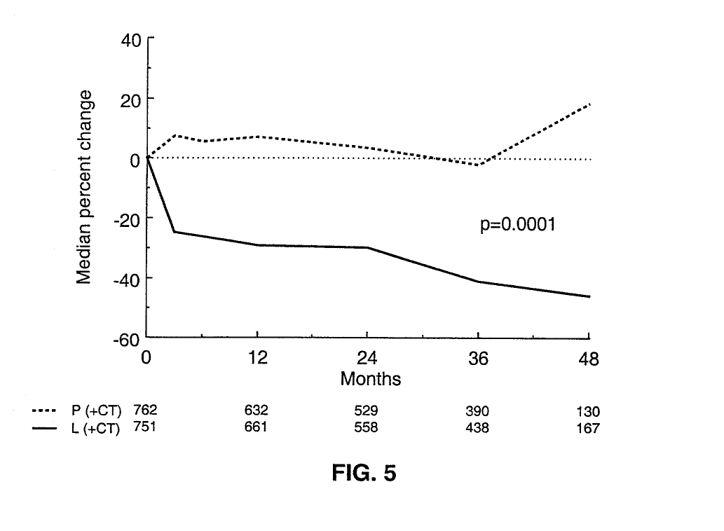

Losartan (plus conventional therapy) also led to an average reduction in proteinuria (urine albumin:creatinine ratio) of 35%, whereas in the placebo group (plus conventional therapy), this ratio tended to increase (p=0.0001, overall treatment effect between groups) (FIG. 5). Losartan reduced the rate of decline in renal function, as assessed by the reciprocal of serum creatinine concentration (median slope −0.056 dl/mg/yr with losartan versus −0.069 with placebo, p=0.01). Thus, losartan slowed the rate of loss of renal function by 18% relative to placebo.

### Subgroup Analyses

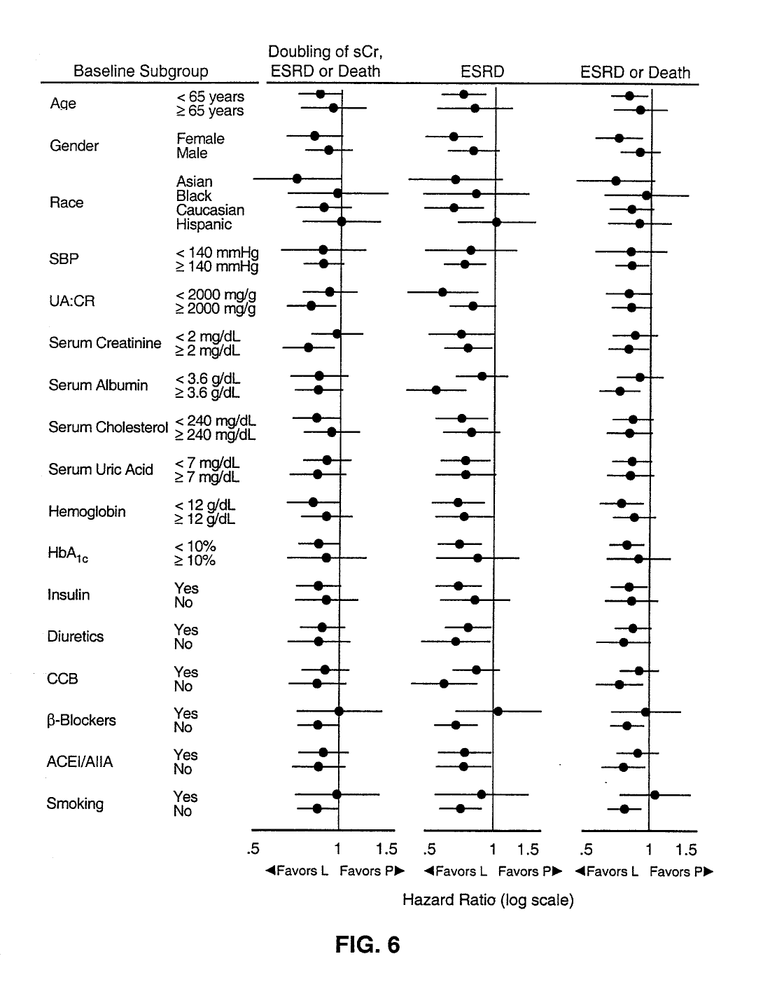

The effect of losartan on the primary composite endpoint was largely reproduced among the many pre-defined subgroups shown in FIG. 6. The effects of losartan on end stage renal disease and combined end stage renal disease or death endpoints also showed consistent benefits among the various predefined subgroups. Losartan appeared to be particularly beneficial in patients with baseline serum creatinine concentration and proteinuria (urine albumin:creatinine ratio) greater than 2.0 mg/dL (177 μmol/L) and 2000 mg/g (226 mg/mmol), respectively. Also, Caucasians and Asians derived considerable benefit from losartan. Furthermore, baseline use of calcium channel antagonists did not alter the beneficial response to losartan.

What is claimed is:

1. A method of preventing end stage renal failure in patients with impaired renal function comprising administration of a therapeutically effective amount of an angiotensin II antagonist or a composition containing an angiotensin II antagonist.

2. The method as recited in claim 1, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl)[1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo [1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

3. The method as recited in claim 2, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

4. The method as recited in claim 1, wherein the composition containing an angiotensin II antagonist includes hydrochlorothiazide.

5. The method as recited in claim 1, wherein the angiotensin II antagonist is losartan potassium or a composition containing losartan potassium.

6. The method as recited in claim 1, wherein the patient is a diabetic patient.

7. The method as recited in claim 6, wherein the patient is a Type II diabetic patient.

8. The method as recited in claim 1, wherein the patient is a renal transplant patient.

9. A method of delaying the progression of renal disease in patients with impaired renal function comprising administration of a therapeutically effective amount of an angiotensin II antagonist or a composition containing an angiotensin II antagonist.

10. The method as recited in claim 8, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6pyrimidinyl]methyl]-3-thiophencarboxylate, 5-[(3,5-dibutyl-1H-1,2,4-triazol-1-yl)methyl]-2-[2-( 1H-tetrazol-5-ylphenyl)]pyridine, 6-butyl-2-(2-phenylethyl)-5-[[2′-(1H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

11. The method as recited in claim 10, wherein the angiotensin II (ATI) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

12. The method as recited in claim 9, wherein the composition containing an angiotensin II antagonist includes hydrochlorothiazide.

13. The method as recited in claim 9, wherein the angiotensin II antagonist is losartan potassium or a composition containing losartan potassium.

14. The method as recited in claim 9, wherein the patient is a diabetic patient.

15. The method as recited in claim 14, wherein the patient is a Type II diabetic patient.

16. The method as recited in claim 9, wherein the patient is a renal transplant patient.

17. A method of slowing the development of end stage renal disease in patients with nephropathy comprising administration of a therapeutically effective amount of an angiotensin II antagonist or a composition containing an angiotensin II antagonist.

18. The method as recited in claim 17, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

19. The method as recited in claim 18, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

20. The method as recited in claim 17, wherein the composition containing an angiotensin II antagonist includes hydrochlorothiazide.

21. The method as recited in claim 17, wherein the angiotensin II antagonist is losartan potassium or a composition containing losartan potassium.

22. The method as recited in claim 17, wherein the patient is a diabetic patient.

23. The method as recited in claim 22, wherein the patient is a Type II diabetic patient.

24. The method as recited in claim 17, wherein the patient is a renal transplant patient.

25. A method of reducing hospitalization for heart failure in patients without clinical signs of heart failure, and who have impaired renal function comprising administration of a therapeutically effective amount of an angiotensin II antagonist or a composition containing an angiotensin II antagonist.

26. The method as recited in claim 25, wherein the angiotensin II (AT1) antagonist is selected from candesartan cilexetil, eprosartan, irbesartan, losartan, tasosartan, telmisartan, valsartan, 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]-4-yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-( 1H-tetrazol-5-ylphenyl)]pyridine, 6-butyl-2-(2-phenylethyl)-5-[[2′-(1H-tetrazol-5-yl) [1,1′-biphenyl]-4-methyl]pyrimidin-4-(3H)-one D,L lysine salt, 5-methyl-7-n-propyl-8-[[2′-(1H-tetrazol-5-yl)biphenyl-4-yl]methyl]-[1,2,4]-triazolo [1,5-c]pyrimidin-2(3H)-one, 2,7-diethyl-5-[[2′-(5-tetrazolyl)biphenyl-4-yl]methyl]-5H-pyrazolo [1,5-b][1,2,4]triazole potassium salt, 2-[2-butyl-4,5-dihydro-4-oxo-3-[2′-(1H-tetrazol-5-yl)-4-biphenylmethyl]-3H-imidazol [4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl-4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl-4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl-4-yl]methyl-1,3,4-thiazoline-2-ylidene]aminocarbonyl-1-cyclopentencarboxylic acid dipotassium salt, and 2-butyl-4-[N-methyl-N-(3-methylcrotonoyl)amino]-1-[[2′-(1H-tetrazol-5-yl)biphenyl-4-yl]methyl]-1H-imidazole-5-carboxylic acid 1-ethoxycarbonyloxyethyl ester, or pharmaceutically acceptable salts thereof.

27. The method as recited in claim 26, wherein the angiotensin II (AT1) receptor antagonist is selected from the group consisting of: candesartan cilexetil, eprosartan, irbesartan, losartan, tasosartan, telmisartan, valsartan, 2-butyl-4-chloro-1-[(2′-tetrazol-5-yl)biphenyl-4-yl)methyl]imidazole-carboxylic acid and 3-(2′-(tetrazol-5-yl)-1,1′-biphen-4-yl)methyl-5,7-dimethyl-2-ethyl-3H-imidazo[4,5-b]pyridine, or pharmaceutically acceptable salts thereof.

28. The method as recited in claim 25, wherein the composition containing an angiotensin II antagonist includes hydrochlorothiazide.

29. The method as recited in claim 25, wherein the angiotensin II antagonist is losartan potassium or a composition containing losartan potassium.

30. The method as recited in claim 25, wherein the patient is a diabetic patient.

31. The method as recited in claim 30, wherein the patient is a Type II diabetic patient.

32. The method as recited in claim 25, wherein the patient is a renal transplant patient.
